(19) United States
(12) Patent Application Publication
(10) Pub. No.: US 2024/0279567 A1
(43) Pub. Date: Aug. 22, 2024

(54) PROCESS FOR PREPARING A GEL COMPOSITION

(71) Applicant: BUNGE NÖVÉNYOLAJIPARI ZÁRTKÖRUEN MUKÖDO RÉSZVÉNYTÁRSASÁG, Budapest (HU)

(72) Inventors: Ágnes SOSNA-SÁRDI, Budapest (HU); Daniel COSSUTA, Budapest (HU); Andrea RADNÓTI, Budapest (HU); Zsolt KEMENY, Budapest (HU)

(73) Assignee: BUNGE NÖVÉNYOLAJIPARI ZÁRTKÖRUEN MUKÖDO RÉSZVÉNYTÁRSASÁG, Budapest (HU)

(21) Appl. No.: 18/571,557

(22) PCT Filed: Jun. 17, 2022
(86) PCT No.: PCT/EP2022/066536

§ 371 Date: Dec. 18, 2023

(30) Foreign Application Priority Data
Jun. 18, 2021 (EP) 21180306.9

(51) Int. Cl. C11B 3/14; A23D 9/013; A23D 9/04; A61K 8/04; A61K 8/92; A61Q 19/00; C11B 3/00; C11B 3/10; C11B 13/00

(52) U.S. Cl. CPC C11B 3/14; A23D 9/013; A23D 9/04; A61K 8/042; A61K 8/922; A61Q 19/00; C11B 3/006; C11B 3/10; C11B 13/00; A61K 2800/10

(57) ABSTRACT

A process for preparing a gel composition including the following steps: (a) providing a composition including an oil and a plant-derived wax where the composition includes from 0.5% to 40.0% by weight of the plant-derived wax; (b) degumming the composition; and (c) deodorizing the degummed composition at a temperature range of from 200° C. to 270° C.

This invention relates to a process for preparing a gel composition, a gel composition obtained by said process and use of gel composition thereof.

### BACKGROUND

Fats and oils are important ingredients of food products and used extensively in the food industry. Crude vegetable oils, which are commonly used, generally contain waxes that may cause turbidity in the final oil product. It is thus necessary to remove said waxes from oils before they are incorporated in food product.

To remove waxes from oils, winterization (type of fractionation) processes are generally implemented. Such process consist in using solvent and/or cooling to separate oil and the waxes present in the oil.

The removed waxes in general contains oxidated materials from crude oil and other impurities associated with unpleasant organoleptic properties and therefore and can hardly be used, in particular for food or cosmetic application. Said waxes may be used in feed or technical oil application and thus have limited value. For example, WO 2014/048943 discloses a process for regenerating filter aid used in a winterization process. The regenerating oil has a relatively high amount of wax content. However, it has not been disclosed how to valorize the wax-containing regenerating oil nor any indication about the possible process to start from it in order to prepare a gel composition.

In addition, winterization process also results in various processed oil waste streams containing wax such as the flushing stream or the stream used to regenerate filter aids applied in oil winterization process. These streams contains relatively high amount of wax, however the wax is in general separated from the oil without proper structure such as gelation. Thus, there is no particular industrial use of these waste streams.

It is generally known that sunflower waxes have excellent gelation properties as disclosed in Winkler-Moser et al., J Am Oil Chem Soc (2019) Vol. 96. Gelation is a key functional property of waxes in food applications such as oleogel, and in cosmetic applications e.g. lipsticks. Nonetheless, for the reasons stated above, such waxes or wax-containing compositions need to be treated to form desired gel structure which requires complex processes such as interesterification.

There remains a need to provide a simple and efficient process to valorize and treat the wax-containing stream generated during the winterization process or other process in order to obtain a gel composition having a desirable appearance and outstanding consistency without unpleasant odor suitable for food applications or cosmetic applications.

### DESCRIPTION OF THE INVENTION

According to the present invention, there is provided a process for preparing a gel composition comprising the following steps:
(a) providing a composition comprising an oil and a plant-derived wax where the composition comprises from 0.5% to 40.0% by weight of the plant-derived wax;
(b) degumming the composition; and
(c) deodorizing the degummed composition at a temperature range of from 200° C. to 270° C.

The process according to the invention has been found to be particularly useful for purifying a wax-containing waste oil stream such as obtained in the winterization process to obtain a gel composition having improved structure and good appearance properties (color, glossy etc.) suitable for food applications or cosmetic applications. It is believed that thanks to the process as defined according to the invention, a gel composition could be properly formed and prepared with the desired functionalities and properties. Meanwhile, the process according to the invention is relatively simple and efficient which does not involve any complex process step such as interesterification.

In the context of the present invention:
the term “degumming” refers to a process for removal of phosphatides from oils, in particular crude vegetable oil in order to improve physical stability and facilitate the further oil processing. Typical degumming process is such as water degumming, acid degumming, alkaline degumming and enzymatic degumming;
the term “deodorizing” or “deodorization” refers to a stripping process by using a stripping agent at a high temperature and at low pressure as known in the art. The stripping agent is typical steam but can also be inert gas, such as nitrogen. It is also understood as steam distillation at a high temperature under reduced pressure. The deodorizing may be carried out in a batch method, a semi-continuous method or a continuous method;
the term “gel” refers to nonfluid colloidal network or polymer network that is expanded throughout its whole volume by a fluid. Gel composition exhibits no flow when in the steady-state. In general, it displays thixotropy and is apparently solid, jelly-like material;
the term “oil” refers to glyceride fats and oils containing fatty acid acyl groups and does not imply any particular melting point. The term “fat” is used synonymously with “oil”;
the term “plant-derived wax” refers to any ester of long chain fatty acids and long chain alcohol that are isolated from plant and naturally present in the oil and can cause turbidity in said oil;
the term “water degumming” refers to any degumming in presence of water. The water degumming may be carried out using a degumming agent selected from a group consisting of citric acid, phosphoric acid or mixture thereof. Preferably, the degumming agent in the water degumming is citric acid. The degumming agent is typically used in a solution form. A basic compound or solution such as sodium hydroxide solution may be added during the degumming process in order to adjust the pH in the aqueous phase; and
the term “waxes” refers to high-melting fatty acid esters of long-chain fatty alcohols such as esters of long chain fatty acids containing 20 to 28 carbon atoms and long chain fatty alcohols containing 22 to 30 carbon atoms.

The wax content in oil mixture may be measured based on ISO/TS 23647—Vegetable fats and oils—Determination of wax content by gas chromatography. Alternatively, the wax content in oil mixture may also be estimated based on saponification value (SV) or saponification number (SN) in conjunction with acid value, in particular when the wax content is high, such as higher than 20% by weight.

Saponification value or saponification number represents the number of milligrams of potassium hydroxide (KOH) required to saponify one gram of fat under the conditions specified in standard methods such as ASTM D1387-89 Standard Test Method for Saponification Number (Empirical) of Synthetic and Natural Waxes. It is a measure of the number of fatty acid residues, and the average molecular weights of each compounds, that contain saponifiable fatty acids present in the sample. These saponifiable compounds are for example: triglycerides, wax esters and free fatty acids as main components of waxy samples. The higher the saponification value is, the more and shorter fatty acid residues are present in the sample in free, or in bonded form by ester bonds.

A model calculation was used to convert saponification value of samples to wax content. The model calculates with the three main components of wax samples: triglycerides, wax esters and free fatty acids. The model neglects unsaponifiable compounds, as unsaponifiable content of sunflower oil is low: 1.5 g/kg (Codex Alimentarius, Section 2. Codex Standards for Fats and Oils from Vegetable Sources CODEX STAN 210-1999), and these compounds are soluble in triglycerides, they remain solved in triglycerides during dry fractionation steps, and do not concentrate together with waxes.

The theoretical saponification value (SV) of pure compounds (triglycerides, waxes and free fatty acids) can be calculated by the following equation (based on AOCS Cd 3a-94 Calculated Saponification Value):

$$SV = Nr of fatty acid residues \times 1000 \times \frac{56.1}{{MW}_{compound}}$$

wherein:
1000=conversion factor mg/g;
56.1=molecular weight of KOH g/mol; and
   MW_{compound}=the average molecular weight of pure compound.

The present invention relates to a process for preparing a gel composition comprising a first step (a) of providing a composition comprising an oil and a plant-derived wax where the composition comprises from 0.5% to 40.0% by weight of the plant-derived wax. Preferably, the present invention relates to a process for preparing a gel composition as defined above in which step (a) is conducted under the following conditions, taken individually or in combination:
the composition comprises from 0.8% to 15.0% by weight of the plant-derived wax, preferably from 1.0% to 10.0% by weight of the plant-derived wax, more preferably from 1.2% to 8.0% by weight of the plant-derived wax and most preferably from 1.5% to 7.0% by weight;
the oil is a vegetable oil. Preferably, the oil is selected from a group consisting of sunflower oil, safflower oil, high oleic sunflower oil, high stearic sunflower oil, olive oil, corn oil, palm oil, coconut oil, palm kernel oil, peanut oil, sesame oil, cottonseed oil, rapeseed oil, canola oil, maize oil, rice bran oil, fraction thereof and mixture thereof. More preferably, the oil is sunflower oil; and/or
the plant-derived wax is selected from a group consisting of sunflower oil wax, rice bran wax, corn oil wax, maize oil wax, olive oil wax, high oleic sunflower oil wax and mixture thereof. More preferably, the plant-derived wax is sunflower oil wax.

In a preferred embodiment, the composition used in step (a) of the process according to the present invention comprises an oil selected from a group consisting of sunflower oil, safflower oil, high oleic sunflower oil, high stearic sunflower oil, olive oil, corn oil, palm oil, coconut oil, palm kernel oil, peanut oil, sesame oil, cottonseed oil, rapeseed oil, canola oil, maize oil, rice bran oil, fraction thereof and mixture thereof and a plant-derived wax selected from a group consisting of sunflower oil wax, rice bran wax, corn oil wax, maize oil wax, olive oil wax, high oleic sunflower oil wax and mixture thereof where the composition comprises from 0.8% to 15.0% by weight of the plant-derived wax.

In a more preferred embodiment, the composition used in step (a) of the process according to the present invention comprises sunflower oil and sunflower oil wax where the composition comprises from 1.0% to 10.0% by weight of sunflower oil wax.

In a still preferred embodiment, the composition used in step (a) of the process according to the present invention comprises sunflower oil and sunflower oil wax where the composition comprises from 1.2% to 8.0% by weight of sunflower oil wax.

In most preferred embodiment, the composition used in step (a) of the process according to the present invention comprises sunflower oil and sunflower oil wax where the composition comprises from 1.5% to 7.0% by weight of sunflower oil wax.

The present invention relates to a process for preparing a gel composition comprising a second step (b) of degumming the composition. Preferably, the present invention relates to a process for preparing a gel composition as defined above in which step (b) is conducted under the following conditions, taken individually or in combination:
degumming is water degumming. Preferably, water degumming is carried out at a temperature of from 80° C. to 95° C.; and/or
degumming step is followed by adsorption carried in a single stage or in multiple stages. The adsorption can be carried out by any appropriate adsorbent. Preferably, the adsorption is carried out using a hydrophilic adsorbent and more preferably using silica as adsorbent. The adsorption is preferably carried out using from 0.5% to 5% by weight of adsorbent, more preferably from 1% to 4% by weight and even more preferably from 1.5% to 3% by weight. The adsorption is preferably carried out at a temperature range of from 50° C. to 120° C., more preferably from 60° C. to 100° C. and even more preferably from 70° C. to 90° C.

In a preferred embodiment, step (b) of the process according to the invention comprises water degumming carried out at a temperature range of from 80° C. to 95° C. using a degumming agent selected from a group consisting of citric acid, phosphoric acid or mixture thereof followed by adsorption carried out at a temperature range of from 50° C. to 120° C. using from 0.5% to 5% by weight of a hydrophilic adsorbent.

In a more preferred embodiment, step (b) of the process according to the invention comprises water degumming carried out at a temperature range of from 80° C. to 95° C. using citric acid as degumming agent followed by adsorption carried out at a temperature range of from 60° C. to 100° C. using from 1% to 4% by weight of silica as adsorbent.

In most preferred embodiment, step (b) of the process according to the invention comprises water degumming carried out at a temperature range of from 80° C. to 95° C. using citric acid as degumming agent followed by adsorption carried out at a temperature range of from 70° C. to 90° C. using from 1.5% to 3% by weight of silica as adsorbent.

The present invention relates to a process for preparing a gel composition comprising a third step (c) of deodorizing the degummed composition at a temperature range of from 200° C. to 270° C. Preferably, the present invention relates to a process for preparing a gel composition as defined above in which step (c) is conducted under the following conditions, taken individually or in combination:
the deodorization is carried out at a temperature range of from 200° C. to 250° C., preferably of from 205° C. to 240° C., more preferably of from 210° C. to 235° C.;
the deodorization is preferably carried out for a period of at most 4 hours, more preferably of at most 3 hours, even more preferably of from 5 minutes to 2 hours, and most preferably of from 15 minutes to 90 minutes; and/or
the deodorization is carried out under a reduced pressure of at most 5 mbar, preferably of at most 4 mbar, and more preferably of from 1 mbar to 3 mbar.

In a preferred embodiment, step (c) of the process according to the invention is carried out at a temperature range of from 200° C. to 250° C. for a period of at most 4 hours under a reduced pressure of at most 5 mbar.

In a more preferred embodiment, step (c) of the process according to the invention is carried out at a temperature range of from 205° C. to 240° C. for a period of at most 3 hours under a reduced pressure of at most 4 mbar.

In an even more preferred embodiment, in step (c) of the process according to the invention is carried out at a temperature range of from 210° C. to 235° C. for a period of from minutes to 2 hours under a reduced pressure of from 1 mbar to 3 mbar.

In most preferred embodiment, the deodorization in step (c) of the process according to the invention is carried out at a temperature range of from 210° C. to 235° C. for a period of from 15 minutes to 90 minutes under a reduced pressure of from 1 mbar to 3 mbar.

Preferably, the present invention relates to a process for preparing a gel composition comprising the following steps:
(a) providing a composition comprising an oil selected from a group consisting of sunflower oil, safflower oil, high oleic sunflower oil, high stearic sunflower oil, olive oil, corn oil, palm oil, coconut oil, palm kernel oil, peanut oil, sesame oil, cottonseed oil, rapeseed oil, canola oil, maize oil, rice bran oil, fraction thereof and mixture thereof and a plant-derived wax selected from a group consisting of sunflower oil wax, rice bran wax, corn oil wax, maize oil wax, olive oil wax, high oleic sunflower oil wax and mixture thereof where the composition comprises from 0.8% to 15.0% by weight of the plant-derived wax;
(b) degumming the composition including water degumming followed by adsorption; and
(c) deodorizing the degummed composition at a temperature range of from 200° C. to 250° C. for a period of at most 4 hours under a reduced pressure of at most 5 mbar.

More preferably, the present invention relates to a process for preparing a gel composition comprising the following steps:
(a) providing a composition comprising sunflower oil and sunflower oil wax where the composition comprises from 1.0% to 10.0% by weight of sunflower oil wax;
(b) degumming the composition including water degumming carried out at a temperature range of from 80° C. to 95° C. using a degumming agent selected from a group consisting of citric acid, phosphoric acid or mixture thereof followed by adsorption carried out at a temperature range of from 50° C. to 120° C. using from 0.5% to 5% by weight of a hydrophilic adsorbent; and
(c) deodorizing the degummed composition at a temperature range of from 205° C. to 240ºC for a period of at most 3 hours under a reduced pressure of at most 4 mbar.

Still preferably, the present invention relates to a process for preparing a gel composition comprising the following steps:
(a) providing a composition comprising sunflower oil and sunflower oil wax where the composition comprises from 1.2% to 8.0% by weight of sunflower oil wax;
(b) degumming the composition including water degumming carried out at a temperature range of from 80° C. to 95° C. using citric acid as degumming agent followed by adsorption carried out at a temperature range of from 60° C. to 100° C. using from 1% to 4% by weight of silica as adsorbent; and
(c) deodorizing the degummed composition at a temperature range of from 210° C. to 235° C. for a period of from 5 minutes to 2 hours under a reduced pressure of from 1 mbar to 3 mbar.

Most preferably, the present invention relates to a process for preparing a gel composition comprising the following steps:
(a) providing a composition comprising sunflower oil and sunflower oil wax where the composition comprises from 1.5% to 7.0% by weight of sunflower oil wax;
(b) degumming the composition including water degumming carried out at a temperature range of from 80° C. to 95° C. using citric acid as degumming agent followed by adsorption carried out at a temperature range of from 70° C. to 80° C. using from 1.5% to 3% by weight of silica as adsorbent; and
(c) deodorizing the degummed composition at a temperature range of from 210° C. to 235° C. for a period of from 15 minutes to 90 minutes under a reduced pressure of from 1 mbar to 3 mbar.

The process according to the present invention may comprise further additional steps. The present invention thus also relates to a process for preparing a gel composition as defined above further comprising at least one of the following steps:
dry fractionation to obtain the solid or semi-solid part for the following steps. Dry fractionation may take place either before or after step (b). Preferably fractionation is conducted at a temperature range of from 20° C. to 40° C., more preferably at a temperature range of from 25° C. to 35° C., even more preferably at a temperature range of from 28° C. to 32° C.; and/or
solvent fractionation (or solvent extraction). Solvent fractionation may take place either before or after step (c). Preferably, the solvent fractionation is carried out by hexane. The solvent fractionation may be carried out in multiple stages in order to obtain a high quality of the product.

The process according to the invention is particularly useful to be combined or integrated with a process for regeneration of spent filter aid during the winterization or a process for direct filtration without any filter aid during the winterization in order to improve the whole winterization process while using the waste regenerating oil stream to prepare a useful and valuable gel composition product.

The invention also relates to a gel composition obtainable or obtained by the process according to the invention.

The invention also relates to use of a gel composition according to the invention in a food application such as margarine or spread or a cosmetic application such as lipstick.

The following non-limiting examples illustrate the invention and do not limit its scope in any way. In the examples and throughout this specification, all percentages, parts and ratios are by weight unless indicated otherwise.

### EXAMPLES

Throughout the following examples:
wax content of waxy oil mixture was measured based on ISO/TS 23647—Vegetable fats and oils—Determination of wax content by gas chromatography;
congealing point was measured based on ASTM D938-05 Standard Test Method for Congealing point of Petroleum Waxes, Including Petrolatum;
peroxide value of waxy oil mixture was measured based on ISO 3960 Animal and vegetable fats and oils—Determination of peroxide value—Iodometric (visual) endpoint determination;
acid value was measured based on ASTM D1386-15 Standard Test Method for Acid Number (Empirical) of Synthetic and Natural Waxes; and
saponification value was measured based on ASTM D1387-89 Standard Test Method for Saponification Number (Empirical) of Synthetic and Natural Waxes.

### Example 1

A sunflower regenerating oil stream was obtained after winterization process by regenerating filter aid as described in EP-A-2900367. The sunflower regenerating oil stream was considered as waste stream of the winterization process and it contained 3.5% by weight of sunflower oil wax.

30.5 kg regenerating oil stream was firstly degummed. The aim of degumming is to remove phosphatides from the regenerating oil stream as phosphatides have large polar groups. The water degumming was performed at 90° C. by adding citric acid 1.60 ml per kg oil citric acid solution (of a concentration of 312 g/l). The mixture was homogenized for 5 min by high shear mixer and gentle mixing was kept for 20 min. Then soda-lye of 1.60 ml per kg oil NaOH solution (of a concentration of 125 g/l) was added. After soda lye addition gentle mixing was kept for 1 min and then 2% by weight of distilled water was added and gentle mixing was kept for another 15 min for hydration of phosphatides. Hydrated phosphatides can be easily separated from the waxy oil stream by centrifuge separator. Since after water degumming the regenerating oil stream might still contain phosphatides and other polar compounds such as soaps, aldehydes and ketones, the degumming was then preferably completed by further adsorption step where these phosphatides and part of the polar components could be adsorbed onto the surface of hydrophilic adsorbent. For the adsorption, 0.2% by weight of water was added first and then 2% by weight of a silica type of adsorbent was added. The mixing was kept for 20 minutes at 75° C. and no vacuum was applied.

The degummed regenerating oil stream was then deodorized in a batch deodorizer (capacity of max. around 30 kg inlet oil). The stream was heated from 100° C. to 210° C. Several samples were taken at different temperatures during the heating. Once the stream reached 210° C., it was kept for 30 min with a stripping steam dosage of 2.5 ml water/kg oil stream/hour and two samples were taken. After 30 min at 210° C., the stream was further heated up to 235° C. and kept for 1.5 hours with a stripping steam dosage of 5.0 ml water/kg oil stream/hour and two samples were taken. The pressure during the deodorization was kept from 1 mbar to 3 mbar. The congealing point, acid value and peroxide value were measured in each sample and a visual evaluation for each sample was carried out as well.

The results are reported in Table 1.

TABLE 1

| | | Congealing | Acid | Peroxide | |
|---|---|---|---|---|---|
| | Sampling | point | Value | Value | |
| Sample | moment | (° C.) | (mg KOH/g) | (meqO2/kg) | Visual evaluation |
| 1 | at 100° C. | 56.7 | 0.06 | 14.81 | Clear separation |
| | | | | | observed between the |
| | | | | | waxy sediment and oily |
| | | | | | supernatant |
| 2 | at 151° C. | 57.6 | 0.04 | 4.46 | Separation less |
| | | | | | pronounced but |
| | | | | | maintaining semi-solid |
| | | | | | structure; no |
| | | | | | homogenous gel |
| | | | | | formation |
| 3 | at 196° C. | 59.0 | 0.06 | 0.32 | Separation less |
| | | | | | pronounced but |
| | | | | | maintaining semi-solid |
| | | | | | structure; no |
| | | | | | homogenous gel |
| | | | | | formation |
| 4 | at 210° C. | 59.1 | 0.04 | 0.24 | No separation |
| | after 15 | | | | observed; homogenous |
| | min | | | | gel formation; glossy |
| | | | | | oleogel; light color |
| 5 | at 210° C. | 59.3 | 0.06 | 0.27 | No separation |
| | after 30 | | | | observed; homogenous |
| | min | | | | gel formation; glossy |
| | | | | | oleogel; light color |
| 6 | at 235° C. | 59.3 | 0 | <0.10 | No separation |
| | after 30 | | | | observed; homogenous |
| | min | | | | gel formation; glossy |
| | | | | | oleogel; light color |
| 7 | at 235° C. | 59.2 | 0 | <0.10 | No separation |
| | after 1.5 | | | | observed; homogenous |
| | hours | | | | gel formation; glossy |
| | | | | | oleogel; light color |

It could be observed that the process according to the invention allows to produce a gel composition with a very low acid value and peroxide value, light color appearance and no unpleasant odor having a desirable gel structure and consistency (oleogel) which makes the product particularly suitable for cosmetic applications or food applications where a structuring oil by wax is required.

### Example 2

The hot regenerating oil stream with a wax content of 2.1% was dry fractionated, where the stream was conditioned at 28-32° C. and then mechanically pressed by filter press.

The saponification value of the solid or semi-solid part retained by the filter was 163.7 mg KOH/g, corresponding to a wax content of approximatively 24.9% (see the calculation demonstrated in Table 2).

TABLE 2

| | | Wax | Free Fatty |
|---|---|---|---|
| | Triglycerides | esters | Acids |
| Number of fatty acid residues | 3 | 1 | 1 |
| (free or bonded in ester bonds) | | | |
| Conversion factor (mg/g) | 1000 | 1000 | 1000 |
| Molecular weight of KOH | 56.1 | 56.1 | 56.1 |
| (g/mol) | | | |
| Average molecular weight | 877 | 710 | 280 |
| of compounds (g/mol) | | | |
| Saponification value of pure | 191.8* | 79.0** | 200.4* |
| compounds (mg KOH/g) | | | |
| Acid value of the composition (i.e. | — | — | 2.1 |
| saponification value of free fatty | | | |
| acids part in the composition) | | | |
| (mg KOH/g) | | | |
| Measured saponification value of | 163.7 | | |
| waxy sample (mg KOH/g) | | | |

Calculations

| % of each compounds (%) | 100 − x − y | x | y |
|---|---|---|---|
| Saponification value of the | 163.7 = ((100 − x − y)/100)*191.8 + (x/100)*79.0 + | | |
| composition | (y/100)*2.10 | | |
| Calculation of “y” (free fatty acids | y = 2.1/200.4 = 1.05 | | |
| level in the composition) (%) | | | |
| Calculation of “x” (waxes level in | x = 24.9 | | |
| the composition) (%) | | | |
| Calculated composition (%) | 74.05 | 24.9 | 1.05 |

*Average molecular weights of triglycerides and free fatty acids of sunflower oil were calculated based on the fatty acid composition of sunflower oil according to Codex Alimentarius, Section 2. Codex Standards for Fats and Oils from Vegetable Sources CODEX STAN 210-1999.
**Average molecular weight of sunflower wax esters were calculated based on the measurement of crude sunflower wax composition of a benchmark sample available on market.

The solid part was then collected and degummed.

The degumming was performed at 90° C. by adding citric acid 5.61 ml per kg oil, citric acid solution (of a concentration of 312 g/l). The mixture was homogenized for 5 min by high shear mixer, then 5.61 ml of soda-lye (concentration of 125 g/l) per kg oil was added and 3.50% hot water was dosed to the waxy oil, then gentle mixing was kept for 1.5 hours. Hydrated phosphatides can be easily separated from the waxy oil stream by centrifuge separator. Since after degumming the waxy oil stream might still contain phosphatides and other polar compounds, the degumming was then preferably completed by further multi-stage adsorption. In the first adsorption step, 2.50% by weight of silica type of adsorbent was added, the mixing was kept for 30 minutes at 80° C. and no vacuum was applied. The spent adsorbent was filtered out of the waxy oil mixture at 90° C. In the second adsorption step 0.20% water was added first and then 2.0% by weight of a silica type of adsorbent was added. The mixing was kept for 30 minutes at 80° C. and no vacuum was applied. Filtration was performed at 90° C.

The degummed oil stream was then collected and deodorized in a batch deodorizer. The degummed oil was heated up to 220° C., it was kept for 30 min with a stripping steam dosage of 2.5 ml water/kg oil stream/hour. After 30 min at 220° C., the stream was further heated up to 235° C. and kept for 1.5 hours with a stripping steam dosage of 5.0 ml water/kg oil stream/hour. The pressure during the deodorization was kept from 1 mbar to 3 mbar.

The free fatty acid content and the peroxide value were almost zero in the deodorized product and the deodorized product had a good glossy gel structure.

The deodorized product was then fractionated by hexane in two stages. Firstly, the deodorized product was mixed with fresh hexane in a ratio of 1:10 (weight of deodorized product (g) to volume of hexane (ml)). The mixture was heated up to 55° C. in order to obtain a homogenous solvent solution. Then the mixture was cooled slowly down to 25° C. The crystallized solid part were separated from the solvent solution by centrifuge or by filtration.

Secondly, the solid part obtained from the first stage which contains oil, wax and hexane was mixed with fresh hexane in the same ratio of 1:10 (weight of deodorized product (g) to volume of hexane (ml)). The mixture was again heated up to 55° C. and slowly cooled down to 25° C. The solid part was separated by centrifuge or filtration and the hexane was removed by evaporation from the solid part in order to obtain a concentrated wax product with a saponification value of no more than 90 (corresponding to a wax content of more than 90%). The solvent supernatant after separation could be used instead of fresh hexane in the first stage of hexane treatment in order to minimize hexane consumption.

The obtained product has been analyzed in comparison to reference samples which are the benchmark wax product available on the market. The results are reported in Table 3.

TABLE 3

| | | Sapon- | Wax content % | |
|---|---|---|---|---|
| | Acid | ification | (calculated | |
| | Value | value | based on sapon- | Congealing |
| | (mg | (mg | ification | point |
| Sample | KOH/g) | KOH/g) | value) | (° C.) |
| Product | 0.26 | 79.4 | 99.7 | 77.0 |
| obtained in | | | | |
| Example 2 | | | | |
| Benchmark | 1.56 | 101.9 | 79.7 | 76.0 |
| product 1 | | | | |
| available on | | | | |
| the market | | | | |
| (Reference 1) | | | | |
| Benchmark | 1.52 | 92.6 | 88.0 | 75.0 |
| product 2 | | | | |
| available on | | | | |
| the market | | | | |
| (Reference 2) | | | | |

As shown in the above results, the product obtained by the process according to the invention has lower acid value, lower saponification value which corresponds to a high purity of wax and higher congealing point which make it more suitable for food applications and cosmetic applications. In particular, the obtained composition with low saponification value provides a cosmetic product with desired structure properties and exceptionally long shelf life.

### Example 3

### Gel Composition According to the Invention

A sunflower oil stream contains 3.3% by weight of sunflower oil wax. 5260 g of this oil stream was heated up to 75° C. For the degumming, 0.20% by weight of water was added together with 2.0% by weight of TriSyl® 300 Silica and no vacuum was applied. The mixture was kept stirred at 75° C. for 30 minutes and then filtered with 0.4% by weight of Clarcel® filter aid at the end of the process. The polar compounds were removed from the oil stream by adsorption onto the surface of TriSyl® 300 Silica.

After filtration, 1660 g degumming oil stream was deodorized. It was heated up to 210° C. first and kept for 30 minutes with a stripping steam dosage of 2.5 ml water/kg oil stream/hour. After 30 min at 210° C., the stream was further heated up to 235° C. and kept for 1.5 hours with a stripping steam dosage of 5.0 ml water/kg oil stream/hour. The pressure during the deodorization was kept around 2 mbar. A gel composition according to the invention was then obtained.

### Comparative Composition

In order to compare the physical properties, a comparative composition was prepared by simply blending and homogenizing 10 g sunflower wax and 300 g refined sunflower to obtain a comparative product with 3.3% by weight of sunflower oil wax.

### Hardness Comparison

The texture property of the gel obtained according to the invention and of the comparative composition was determined by measuring the hardness using Brookfield texture analyzer with a penetrometer probe of 6 mm diameter (penetration depth of 5 mm at 0.5 mm/sec).

The force to penetrate the gel composition according to the invention increased from the beginning and stabilized around 3.2N.

On the contrary, the force to penetrate the comparative composition decreased from the beginning and stabilized around 1.5N.

It is thus shown that the gel composition according to the invention has a harder texture and higher stress resistance compared to the comparative composition.

### Viscoelasticity Comparison

The viscoelasticity property of the gel obtained according to the invention and of the comparative composition was also investigated by using a parallel-plate geometry of 40 mm diameter serrated plate at 20° C. when the increasing oscillated stress was applied.

In a range of 0.1 Pa to 50 Pa of oscillated stress, for instance, at an oscillated stress level of 1 Pa, the gel composition according to the invention presented the elastic modulus (G′) of around 300000 Pa and the viscous modulus (G″) of around 60000 Pa. On the contrary, the comparative composition presented the elastic modulus (G′) of around 100000 Pa and the viscous modulus (G″) of around 30000 Pa.

Furthermore, in the gel composition according to the invention, the elastic modulus (G′) remained higher than the viscous modulus (G″) at a high oscillated stress level (>100 Pa) while in the comparative composition, the elastic modulus (G′) of the comparative composition became lower than the viscous modulus (G″) at a high oscillated stress level (>100 Pa).

In addition, the elastic modulus (G′) in the comparative composition dropped much faster than the elastic modulus (G′) in the gel composition according to the invention at this high oscillated stress level.

The above results demonstrate that the gel composition according to the invention is different from the comparative composition and has a much better texture and improved shear resistance. These results are also in line with the previous texture analysis.

### CONCLUSION

Therefore, although both the gel composition according to the invention and the comparative composition were prepared from sunflower oil including the same content of sunflower wax, the gel composition prepared by the claimed process has different physical properties than the comparative composition, which presents a harder structure and higher shear resistance.

Those properties are particularly desirable for food applications or cosmetic applications.

1. A process for preparing a gel composition comprising the following steps:
(a) providing a composition comprising an oil and a plant-derived wax where the composition comprises from 0.5% to 40.0% by weight of the plant-derived wax;
(b) degumming the composition; and
(c) deodorizing the degummed composition at a temperature range of from 200° C. to 270° C.

2. The process according to claim 1, wherein, in step (a), the composition comprises from 0.8% to 15.0% by weight of plant-derived wax.

3. The process according to claim 1, wherein in step (a), the oil is selected from a group consisting of sunflower oil, safflower oil, high oleic sunflower oil, high stearic sunflower oil, olive oil, corn oil, palm oil, coconut oil, palm kernel oil, peanut oil, sesame oil, cottonseed oil, rapeseed oil, canola oil, maize oil, rice bran oil, fraction thereof and mixture thereof.

4. The process according to claim 1, wherein in step (a), the plant-derived wax is selected from a group consisting of sunflower oil wax, rice bran wax, corn oil wax, maize oil wax, olive oil wax, high oleic sunflower oil wax and mixture thereof.

5. The process according to claim 1, wherein degumming of step (b) is water degumming.

6. The process according to claim 5, wherein water degumming is carried out using a degumming agent selected from a group consisting of citric acid, phosphoric acid or mixture thereof.

7. The process according to claim 5, wherein adsorption is carried out after water degumming.

8. The process according to claim 7, wherein the adsorption is carried out using a hydrophilic adsorbent.

9. The process according to claim 7, wherein the adsorption is carried out using from 0.5% to 5% by weight of adsorbent.

10. The process according to claim 7, wherein the adsorption is carried out at a temperature range of from 50° C. to 120° C.

11. The process according to claim 1, wherein the deodorization is carried out at a temperature range of from 200° C. to 250° C.

12. The process according to claim 1, wherein the deodorization is carried out for a period of at most 4 hours.

13. The process according to claim 1, wherein the deodorization is carried out under a reduced pressure of at most 5 mbar.

14. The process according to claim 1, wherein dry fractionation is carried out before step (b) to obtain the solid or semi-solid part at a temperature range of from 20° C. to 40° C.

15. The process according to claim 1, wherein the deodorized composition obtained after step (c) is subject to solvent fractionation.

16. A gel composition obtainable by a process according to claim 1.

17. A method comprising applying a gel composition according to claim 16 in a food application or a cosmetic application.
